(19) United States
(12) Patent Application Publication
(10) Pub. No.: US 2021/0134433 A1
(43) Pub. Date: May 6, 2021

(54) SYSTEM AND METHOD FOR MAINTAINING PATIENT ADHERENCE TO A DIETARY PROGRAM WHEN TREATING DIABETES

(71) Applicant: Lysulin, Inc., San Diego, CA (US)

(72) Inventors: John Burd, San Diego, CA (US); Abe Fuentes, San Diego, CA (US)

(21) Appl. No.: 16/673,783

(22) Filed: Nov. 4, 2019

(51) Int. Cl. G16H 20/60; G16H 20/30

(52) U.S. Cl. CPC G16H 20/60; G16H 20/30

(57) ABSTRACT

Methods are disclosed for treating a disorder associated with a disorder. Some methods may include receiving patient demographic information associated with a patient, receiving patient medical information associated with the patient, determining a likelihood of patient adherence for the patient based upon the patient demographic information and the patient medical information, and generating a dietary regimen including one or more meal recommendations consumed by the patient during a time period and an exercise regimen including one or more exercise recommendations performed by the patient during the time period based on the likelihood of patient adherence.

### TECHNICAL FIELD

The present disclosure is generally related to treatment of type II diabetes mellitus, and more specifically, the present disclosure is directed to systems and methods for maintaining patient adherence to a ketogenic diet and exercise.

### BACKGROUND

The current way of life in most urbanized societies may be characterized by less physical work and increased consumption of fat, carbohydrates, and proteins, resulting in the energy intake exceeding energy expenditure. This shift in the energy balance causes the body to store excess energy in the form of fat. A lifestyle characterized by this long-term energy imbalance leads to overweight and increases a risk of obesity. The percentage of overweight people increases year by year and obesity is a disease that is reaching epidemic proportions in some countries. The health risks associated with being overweight and obesity are numerous and it has been shown that these conditions contribute to morbidity and mortality of individuals suffering from diseases such as hypertension, stroke, diabetes mellitus type II, gallbladder disease, and ischemic heart disease. The cosmetic perspective of body fat is also to be considered as the demand for dietary supplements or medicine to gain or maintain a leaner body is constantly increasing.

In particular, diabetes mellitus type II is the most common type of diabetes and accounts for around 90% of all cases of diabetes. It is characterized by hyperglycemia, insulin resistance, and relative insulin deficiency. Specifically, in type II diabetes, the response to insulin, a hormone made by the pancreas that helps glucose get into the cells so that it can be used for energy, is diminished. This is defined as insulin resistance. During this state, insulin is ineffective and is initially countered by an increase in insulin production by the body to maintain glucose homeostasis. Over time, insulin production decreases, resulting in type II diabetes. This is marked by an increase in blood glucose. Blood glucose is the body's main source of energy and comes mainly from consuming foods that are high in carbohydrates. However, in patients with type II diabetes, the cells do not get enough energy despite the increased intake of carbohydrates.

A majority of individuals suffering from type II diabetes are obese with central visceral adiposity. Therefore, the adipose tissue plays a crucial role in the pathogenesis of type II diabetes.

### SUMMARY

In accordance with one or more embodiments, various features and functionality can be provided to enable or otherwise facilitate reversing type II diabetes mellitus.

Embodiments of the disclosure are directed to methods for treating a disorder associated with a disorder. In some embodiments, the method may include receiving patient demographic information associated with a patient. In other embodiments, the method may include receiving patient medical information associated with the patient. In other embodiments, the method may also include determining a likelihood of patient adherence for the patient based upon the patient demographic information and the patient medical information. In other embodiments, the method may further include generating a dietary regimen including one or more meal recommendations consumed by the patient during a time period and an exercise regimen including one or more exercise recommendations performed by the patient during the time period based on the likelihood of patient adherence

In some embodiments, the patient demographic information may include age, gender, and marital status.

In some embodiments, the patient medical information may include at least one medical condition associated with the patient.

In some embodiments, the method may further include obtaining information related to other patients based on the patient demographic information and the patient medical information.

In some embodiments, the determination of likelihood of patient adherence for the patient may be based upon the information related to other patients.

In some embodiments, the determination of the likelihood of patient adherence of the user may be calculated using Bayesian statistics.

In some embodiments, the meal recommendations may include at least one meal from a restaurant.

In some embodiments, a total daily caloric intake of the one or more meal recommendations may be less than a baseline caloric intake for the patient.

In some embodiments, a daily carbohydrate amount of the one or more meal recommendations may be approximately less than 50 grams.

In some embodiments, the disorder may include at least a metabolic syndrome, insulin resistance, and diabetes.

### BRIEF DESCRIPTION OF THE DRAWINGS

FIG. 1 illustrates example systems and a network environment, according to an implementation of the disclosure.

FIG. 2 illustrates an example system configured to generate a diet and exercise program, according to an implementation of the disclosure.

FIG. 3 illustrates an example predictive method for determining a likelihood of the diet and exercise program followed by a patient determined by the system of FIG. 2, according to an implementation of the disclosure.

FIG. 4 illustrates an example process for generating diet and exercise program notifications based on the likelihood of the diet and exercise program being followed by a patient determination of FIG. 3, according to an implementation of the disclosure.

FIG. 5 illustrates an example computing system that may be used in implementing various features of embodiments of the disclosed technology.

### DETAILED DESCRIPTION

Type II diabetes mellitus is a metabolic disease that can be prevented through lifestyle modification, and diet and weight control. While numerous pharmacological therapies have been developed, no cure is available for the disease, despite new insight into its pathophysiology. Management should be tailored to improve the quality of life of individuals with type II diabetes.

One group of pharmacological agents includes biguanides, e.g., metformin commonly used in overweight and obese patients, which suppresses hepatic glucose production, increases insulin sensitivity, enhances glucose uptake, increases fatty acid oxidation, and decreases the absorption of glucose from the gastrointestinal tract.

However, pharmacological therapies do not provide a lasting cure. Rather, the medications lower the blood sugar levels by increasing insulin sensitivity and allowing the absorption of carbohydrates and especially glucose from the blood into liver, fat, and skeletal muscle cells. Over time, these medications lose their ability to increase cellular sensitivity to insulin requiring higher medication doses, and eventually leading to additional agents and then insulin supplementation. Further still, some of these medications actually result in patient weight gain.

Because one of the leading causes for type II diabetes is obesity and lack of exercise, available pharmacological interventions cannot provide sustained long term positive outcomes. Rather, a diet and exercise regimen is a more effective treatment option intended to reverse type II diabetes.

In some embodiments, a ketogenic diet comprising a low-carbohydrate, high fat, moderate protein diet, and supplying approximately 80% of calories from fat, 15% calories from protein, and 5% calories from carbohydrates may be an effective treatment for patients with type II diabetes. In some embodiments, the ketogenic diet may restrict carbohydrate intake usually to less than 50 grams per day.

In some embodiments, a consumption of a ketogenic diet induces a physiological metabolic state of elevated serum ketone bodies known as “ketosis” in which the cellular oxidation of ketone bodies is enhanced. Ketone bodies are short-chained, four-carbon molecules synthesized in liver mitochondria through a process called “ketogenesis.” The ketogenic process requires acetyl-CoA, generated via the beta-oxidation of fatty acids, and continues with the aid of several enzymes. This process ultimately results in the production of the primary ketone bodies to be released into the bloodstream. Ketone bodies are more energy efficient substrates than glucose or fatty acids. Accordingly, in ketosis, ketones function as fuel alternatives to glucose and can help diabetes patients who are unable to utilize glucose effectively, resulting in fat loss.

In some embodiments, a ketogenic diet, as alluded to above, will yield better results when combined with an exercise regimen. For example, exercise improves blood sugar control, decreases body fat content, and decreases blood lipid levels in addition to promoting weight loss. In some embodiments, both aerobic and anaerobic exercise may be utilized. For example, aerobic exercise may lead to an improved insulin sensitivity. Similarly, resistance training is also useful and the combination of both types of exercise may be most effective.

A major limitation to any treatment program that includes a diet and/or exercise components is patient adherence. For example, the success of a type II diabetes reversal program comprising a ketogenic diet and an exercise regimen is largely dependent on how strictly a patient follows it. However, patients following a low-carbohydrate and/or ketogenic diet are among those with the highest incidence of lapses and dropouts. The low adherence rate may be related to comparatively limited food choices and/or requirements to consume foods with reduced taste sensation, palatability, and/or structure. This is especially true when dining out.

Accordingly, various embodiments disclosed herein are directed to systems and methods for providing a patient with a ketogenic diet and exercise program with a high likelihood of adherence. Various embodiments may leverage patients' demographic information, personal preferences, and information related to other similarly situated patients who successfully followed the ketogenic and exercise program when generating diet and exercise determinations.

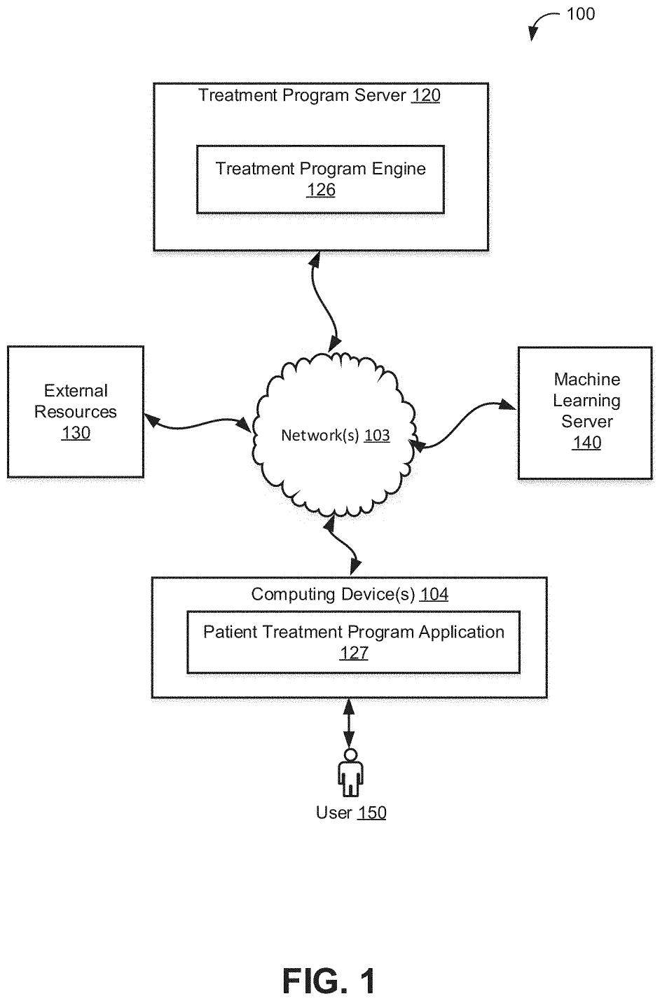

Before describing the technology in detail, it is useful to describe an example system in which the presently disclosed technology can be implemented. FIG. 1 illustrates one such example treatment program system 100.

FIG. 1 illustrates an example treatment program system 100 which allows patients suffering from a medical condition (e.g., type II diabetes) to obtain a diet and exercise program they are likely to follow. As alluded to above, the success of treating and/or reversing type II diabetes by following a ketogenic diet and/or exercise program is predicated on how well the patient actually follows the diet and exercise program. By generating a personalized ketogenic diet and exercise program based on individual patient characteristics, the likelihood of patient adherence is increased. For example, a ketogenic diet and exercise program may be generated by determining a likelihood of a patient of a particular age or gender having a particular medical condition will follow the treatment program.

Additionally, a likelihood a patient will follow the treatment program may be determined based on information related to other patients that successfully adhered to ketogenic diet and exercise program, as described herein. In some embodiments, system 100 may include a treatment program server 120, a machine learning server 140, external resources 130, a one or more client computing devices 104, and a network 103. A user 150 (e.g., a patient) may be associated with client computing device 104 as described in detail below.

In some embodiments, treatment program server 120 may include a processor, a memory, and network communication capabilities. In some embodiments, treatment program server 120 may be a hardware server. In some implementation, treatment program server 120 may be provided in a virtualized environment, e.g., treatment program server 120 may be a virtual machine that is executed on a hardware server that may include one or more other virtual machines. Treatment program server 120 may be communicatively coupled to network 103. In some embodiments, treatment program server 120 may transmit and receive information to and from one or more of client computing devices 104, machine learning server 140, external resources 130, and/or other servers via network 103.

In some embodiments, as alluded to above, treatment program server 120 may include a distributed treatment program engine 126 and a corresponding client treatment program application 127 running on one or more client computing devices 104.

In some embodiments, users of treatment program system 100 (e.g., patients) may access the treatment program engine 126 via client computing device(s) 104. In some embodiments, the various below-described components of FIG. 1 may be used to initiate treatment program application 127 within client computing device 104. In some embodiments, treatment program application 127 may be configured to obtain information related to the patient entered by user 150 and display the diet and exercise program determined by treatment program engine 126. For example, treatment program application 127 may be configured to allow users to enter their name, age, gender, weight, height, occupation, marital status, number of kids, current physical activity level, one or more medical conditions, and/or other similar information. In some embodiments, patients may be required to provide additional information to via one or more follow-up questions based on the information provided, as described in further detail below.

In some embodiments, machine learning server 140 and/or other components of system 100 may be configured to use machine learning, e.g., use a machine learning model that utilizes machine learning to determine a likelihood a patient will adhere to a ketogenic diet and exercise program based on the patient information, as described in further detail below. In some embodiments, machine learning server 140 may include one or more processors and memory and network communication capabilities. In some embodiments, machine learning server 140 may be a hardware server connected to network 103, using wired connections, such as Ethernet, coaxial cable, fiber-optic cable, etc., or wireless connections, such as Wi-Fi, Bluetooth, or other wireless technology. In some embodiments, machine learning server 140 may transmit data between one or more of leads processing server 130, client computing device 104, external resources 130, and/or other components via network 103.

In some embodiments, external resources 130 may comprise one or more dining establishments (e.g., restaurants) that offer meals consistent with ketogenic diet guidelines. In some embodiments, external resources 130 may comprise one or more dining establishments located within a geographic area associated with user 150. For example, dining establishment information may be used by treatment program engine 126 when generating a diet determination, as will be further described in detail below.

In some embodiments, treatment program engine 126 may communicate and interface with a framework implemented by external resources 130 using an application program interface (API) that provides a set of predefined protocols and other tools to enable the communication. For example, the API can be used to communicate particular data from an insurance carrier used to connect to and synchronize with treatment program engine 126.

In some embodiments, client computing device 104 may include a variety of electronic computing devices, such as, for example, a smartphone, tablet, laptop, computer, wearable device, television, virtual reality device, augmented reality device, displays, connected home device, Internet of Things (IOT) device, an enhanced general packet radio service (EGPRS) mobile phone, a media player, a navigation device, a game console, a television, a remote control, or a combination of any two or more of these data processing devices, and/or other devices. In some embodiments, client computing device 104 may present content to a user and receive user input. In some embodiments, client computing device 104 may parse, classify, and otherwise process user input. For example, client computing device 104 may store user input associated with an agent claiming or selecting a lead, as will be described in detail below.

In some embodiments, client computing device 104 may be equipped with GPS location tracking and may transmit geolocation information via a wireless link and network 103. In some embodiments, treatment program server 120 and/or distributed chat application 126 may use the geolocation information to determine a geographic location associated with user 150. In some embodiments, treatment program server 120 may use signal transmitted by client computing device 104 to determine the geolocation of user 150 based on one or more of signal strength, GPS, cell tower triangulation, Wi-Fi location, or other input. In some embodiments, the geolocation associated with user 150 may be used by one or more computer program components associated with treatment program engine 126 during coverage recommendation determination.

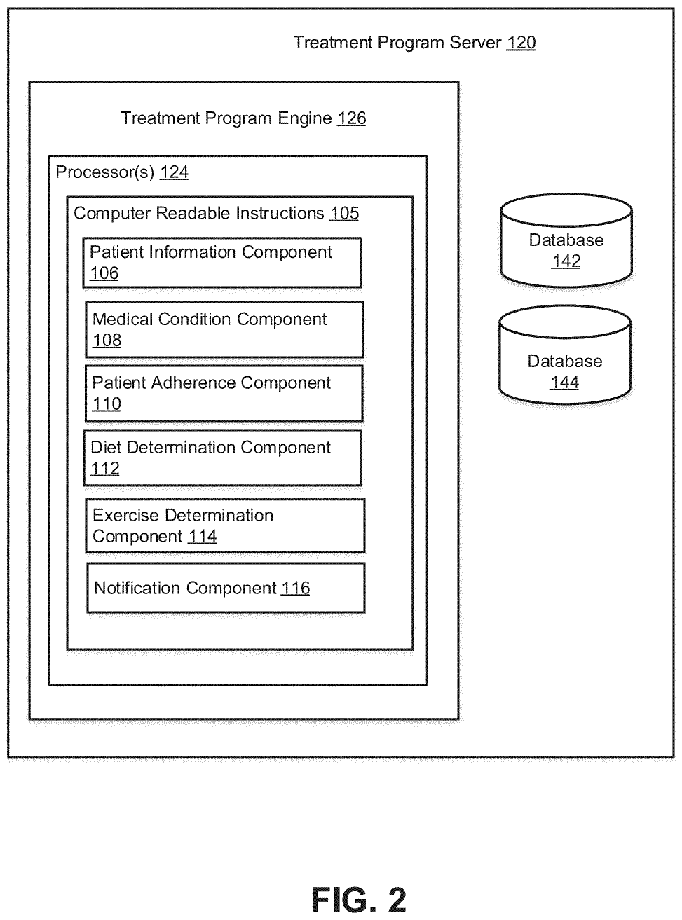

FIG. 2 illustrates an example treatment program server 120 of treatment program system 100 illustrated in FIG. 1 configured in accordance with one embodiment. In some embodiments, the various below-described components of FIG. 2 may be used to determine a diet and exercise program based on specific patient circumstances, as described herein.

In some embodiments, treatment program server 120 may include treatment program engine 126, as alluded to above. In some embodiments, treatment program engine 126 may be operable by one or more processor(s) 124 configured to execute one or more computer readable instructions 105 of one or more computer program components. In some embodiments, the computer program components may include one or more of a patient information component 106, a medical condition component 108, a patient adherence component 110, a diet determination component 112, an exercise regimen determination component 114, a notification component 116, and/or other such components.

In some embodiments, treatment program server 120 may also include one or more databases. For example, databases 142 and 144 may be used to store data used by treatment program engine 126. For example, database 142 may store patient demographic information received via client computing device 104. In some embodiments, database 144 may store demographic information, diet information, and exercise information associated with other patients of treatment program system 100.

In some embodiments, patient information component 106 may be configured to obtain patient demographic information when determining a ketogenic diet and exercise program. For example, patient information component 106 may be configured to obtain information that is being provided by user input via client computing device 104. In some embodiments, patient information may include name, age, gender, weight, height, occupation, marital status, number of kids, current physical activity level, and/or other similar information.

In some embodiments, patient information may include information characterizing the patient's physical activity. For example, the patient may enter the number of times they currently exercise. Alternatively, the patient may provide the number of miles they walk or bike on a weekly basis.

In some embodiments, patient information component 106 may be configured to prompt the user with questions configured to elicit additional information related to their diet and exercise habits. For example, information may include questions clarifying whether the patient will be cooking their meals or dining outside, how much time they spend traveling during a particular period, how likely they are to join a gym, and so on. In some embodiments, patient information component 106 may be configured to generate additional questions based on previously entered information by user 150.

In some embodiments, patient information component 106 may be configured to determine one or more preferences associated with user 150. For example, users' preferences may include cuisine preferences, taste considerations, cost considerations, convenience considerations, time considerations, and other similar considerations.

In some embodiments, patient information component 106, may be configured to use machine learning to determine one or more user preferences, e.g., preferences for meal preparation and consumption, taste preferences, cost, and convenience. For example, a patient who has a demanding job may be less likely to cook at home, while a patient who has a flexible work schedule and grows their own fruits and vegetables may be more likely to prepare meals at home.

In some embodiments, medical condition component 108 may be configured to obtain patient medical diagnosis and/or medical condition information when determining a ketogenic diet and exercise program. In some embodiments, medical diagnosis and/or medical condition information may include details related to type II diabetes mellitus, e.g., time of diagnosis, severity of the condition, current medications used by the patient, blood glucose level, and/or other such relevant data. For example, medical condition component 108 may be configured to obtain information that is being provided by user 150 input via client computing device 104. In some embodiments, medical diagnosis and/or medical condition information may include information related to additional medical conditions the patient may be suffering from.

In some embodiments, patient adherence component 110 may be configured to determine a likelihood that the patient will adhere to a particular diet and exercise program based on the patient information provided by the user to patient information component 106. For example, based on the patient information indicating that the patient has never followed a diet and exercise plan before, has a number of other medical conditions, and leads a sedentary lifestyle, patient adherence component 110 may determine a likelihood of 30 percent that patient will follow a diet and exercise program.

In some embodiments, patient adherence component 110 may be configured to determine a likelihood that the patient will adhere to a particular diet and exercise program by using information related to other users of treatment program system 100. For example, patient adherence component 110 may determine patient adherence based on actual success adhering to a diet related to other patients having similar patient demographics, medical diagnosis information, and other information.

In some embodiments, when determining a likelihood that the patient will adhere to a particular diet and exercise program, patient adherence component 110 may be configured to use one or more of a deep learning model, a logistic regression model, a Long Short Term Memory (LSTM) network, supervised or unsupervised model, etc. In some embodiments, patient adherence component 110 may utilize a trained machine learning classification model. For example, the machine learning may include decision trees and forests, hidden Markov models, statistical models, cache language model, and/or other models. In some embodiments, the machine learning may be unsupervised, semi-supervised, and/or incorporate deep learning techniques.

In some embodiments, diet determination component 112 may be configured to determine one or more dietary plans relevant to the patient's needs based on patient information received by patient information component 106 and a likelihood of patient adherence determination made by patient adherence component 110. For example, diet determination component 112 may provide a ketogenic diet plan for the treatment of type II diabetes. The ketogenic diet may be used for complete nourishment of a patient for a particular time period, e.g., 12 hours. In some embodiments, the ketogenic diet may include one or more meal recommendations, with each meal comprising protein, fat, and carbohydrates. In some embodiments, the total daily amounts of each of protein, fat, and carbohydrates may be determined based on individual patient characteristics. For example, the daily caloric intake and the ratio of protein and fat may be different for patients having different weight. In some embodiments, each meal may be substantially free of carbohydrates. In some embodiments, the effective amount of fat and protein determined by the diet determination component 112 may be an amount that induces weight loss in a patient.

In some embodiments, diet determination component 112 may be configured to determine the ratio of fat to protein in each meal and/or all meals consumed within a particular time period (e.g., one day). For example, the ratio of fat to protein may be in the range of approximately 0.05:1 to about 1:1 by weight. For example, the daily (24-hour) protein amount may be approximately 1.0 g/kg/day to about 3.0 g/kg/day. In other embodiments, the daily protein amount may be in the range of about 1.3 g/kg/day to approximately 2.5 g/kg/day of protein.

In some embodiments, the daily amount of protein may be at least approximately 1 g/kg/day, at least approximately 1.5 g/kg/day, at least approximately 2 g/kg/day, at least approximately 2.5 g/kg/day, at least about 3 g/kg/day, at least about 4 g/kg/day, at least approximately 5 g/kg/day, at least approximately 10 g/kg/day, at least approximately 15 g/kg/day, at least approximately 20 g/kg/day, at least approximately 30 g/kg/day, at least approximately 40 g/kg/day, or at least approximately 50 g/kg/day. In some embodiments, the diet may provide a daily amount of protein in an amount equal to or greater than 60 grams, 80 grams, 100 grams, 110 grams, 120 grams, 130 grams, 140 grams or 150 grams, or any amount in between.

In some embodiments, the daily amount of fat may be in the range of about 0.1 g/kg/day to about 2.5 g/kg/day. In some embodiments, the daily amount of fat may be at least approximately 0.05 g/kg/day, at least approximately 0.1 g/kg/day, at least approximately 0.15 g/kg/day, at least approximately 0.2 g/kg/day, at least approximately 0.5 g/kg/day, at least approximately 1 g/kg/day, at least approximately 1.5 g/kg/day, at least approximately 2 g/kg/day, at least approximately 2.5 g/kg/day, at least approximately 3 g/kg/day, at least approximately 4 g/kg/day, at least approximately 5 g/kg/day, at least approximately 10 g/kg/day, at least approximately 15 g/kg/day, at least approximately 20 g/kg/day, at least approximately 30 g/kg/day, at least approximately 40 g/kg/day, or at least approximately 50 g/kg/day.

In some embodiments, diet determination component 112 may be configured to determine individual meal recommendations based on the daily protein and fat intake amounts, as alluded to above. For example, individual meal recommendations may include a breakfast meal recommendation, a lunch meal recommendation, a dinner meal recommendation, and a snack meal recommendation.

In some embodiments, individual meal recommendations may be based on patient information, e.g., patient preferences. For example, individual meal recommendations generated for a patient that has indicated a preference for Mexican food may include meal recommendations that include a variety of Mexican dishes (e.g., guacamole) or include ingredients associated with Mexican cuisine (e.g., limes, avocados, etc.). In some embodiments, individual meal recommendations generated for patients who have indicated cost or time considerations may include meals that are fast to prepare and only require readily available ingredients. In some embodiments, individual meal recommendations generated for patients who have indicated a preference for dining out may include meals that are available at dining establishments within a particular geographical region. For example, the geographical region may be an area near a patient's home or work, or a destination visited by the patient during a business trip.

In some embodiments, individual meal recommendations may be based on the likelihood of patient adherence determination made by patient adherence component 110. For example, patients that have a low adherence likelihood may include meal recommendations that were favored by other patients with similar demographic and other characteristics.

In some embodiments, diet determination component 112, may be configured to use machine learning to determine one or more meal recommendations.

In some embodiments, diet determination component 112 may generate a daily meal plan that may not induce ketosis. That is, the meal plan may include meal recommendations comprising lower fat and protein amounts by including some carbohydrates. Because most patients will have a hard time eliminating all carbohydrates, having a diet plan that slowly reduces the carbohydrate intake results in a higher rate of patient adherence. Essentially, the daily meal plan may be adjusted until the target protein and fat amounts are achieved.

In some embodiments, diet determination component 112 may generate a daily meal plan that may include vitamins and supplements (e.g., lysulin).

In some embodiments, diet determination component 112 may generate a daily meal plan that may include an intermittent fasting protocol, i.e., a meal plan that includes periods where no food is consumed followed by food consumption periods.

In some embodiments, exercise determination component 114 may be configured to determine one or more exercise plans needs based on patient information received by patient information component 106 and a likelihood of patient adherence determination made by patient adherence component 110.

For example, exercise determination component 114 may provide an exercise plan comprising of exercise recommendations and may be suitable for the management of type II diabetes. The exercise recommendations may include aerobic and anaerobic exercises performed during a particular time period (e.g., a day or a week).

In some embodiments, exercise determination component 114 may be configured to determine daily exercise recommendations based on a weekly exercise goal, as alluded to above. For example, individual exercise recommendations may include a 30 min. walking exercise, a 60 min. biking exercise, a 30 min. strength training exercise, and so on.

In some embodiments, individual exercise recommendations may be based on patient information, e.g., patient preferences. For example, individual exercise recommendations generated for a patient that has indicated a preference for outdoor fitness may include exercise recommendations that can be performed outdoors (e.g., running, sports). In some embodiments, individual exercise recommendations generated for patients who have indicated cost or time considerations may include exercises that can be performed in short increments during the day at a patient's home. In some embodiments, the individual exercise recommendations generated for patients who have indicated a preference for going to the gym may include recommendations of gyms or fitness studios in a particular geographical region. For example, the geographical region may be an area near the patient's home or work, or a destination visited by the patient during a business trip.

In some embodiments, exercise recommendations may be based on a likelihood of patient adherence determination made by patient adherence component 110. For example, patients that have a low adherence likelihood may include exercise recommendations that were favored by other patients with similar demographic and/or other characteristics.

In some embodiments, exercise determination component 114, may be configured to use machine learning to determine one or more exercise recommendations.

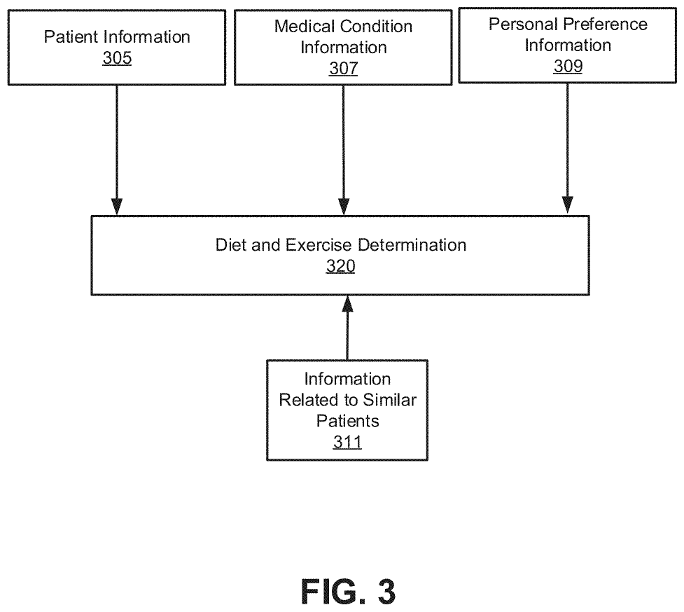

In some embodiments, diet determination component 112 and exercise determination component 114 may be configured to determine one or more meal recommendations and exercise recommendations, respectively, using a number of models or methods. For example, Bayesian-type statistical analysis may be used during the likelihood determination. For example, as illustrated in FIG. 3, determination component 112 and exercise determination component 114 may perform a diet and exercise analysis 320 which may include a Bayesian-type statistical analysis using patient classification information 305, medical condition information 307, personal preference information 309, and information related to other patients 311.

Referring back to FIGS. 1-2, In some embodiments, notification component 116 may be configured to generate one or more notifications or alerts based on diet recommendations determined by diet determination component 112 and exercise recommendations determined by exercise determination component 114. For example, a notification may be generated by notification component 116 to remind the patient to perform a daily physical activity. In some embodiments, notification component 116 may be configured to generate one or more notifications transmitted from treatment program engine 126 via a wireless link and a communications network 103 to client computing device 104 associated with user 150.

In some embodiments, notification component 116 may be configured to generate one or more notifications in response to a patient satisfying a geolocation and/or a time requirement. For example, a geolocation requirement may comprise geographic locations associated with an exercise facility where the patient can perform one or more exercise recommendations generated by exercise determination component 114. Alternatively, a geolocation requirement may comprise geographic locations associated with a restaurant that offers dining options that correspond to one or more diet recommendations generated by diet determination component 112. Similarly, a time requirement may comprise a time (e.g., noon) that corresponds to the timing of one or more exercise recommendations generated by exercise determination component 114 and/or one or more diet recommendations generated by diet determination component 112.

In some embodiments, upon determining user's 150 geolocation satisfies a geolocation requirement, may cause notification component 116 to generate a notification comprising a message informing that user 150 has entered the geolocation associated with an exercise facility where the patient can perform one or more exercise recommendations generated by exercise determination component 114, as alluded to above.

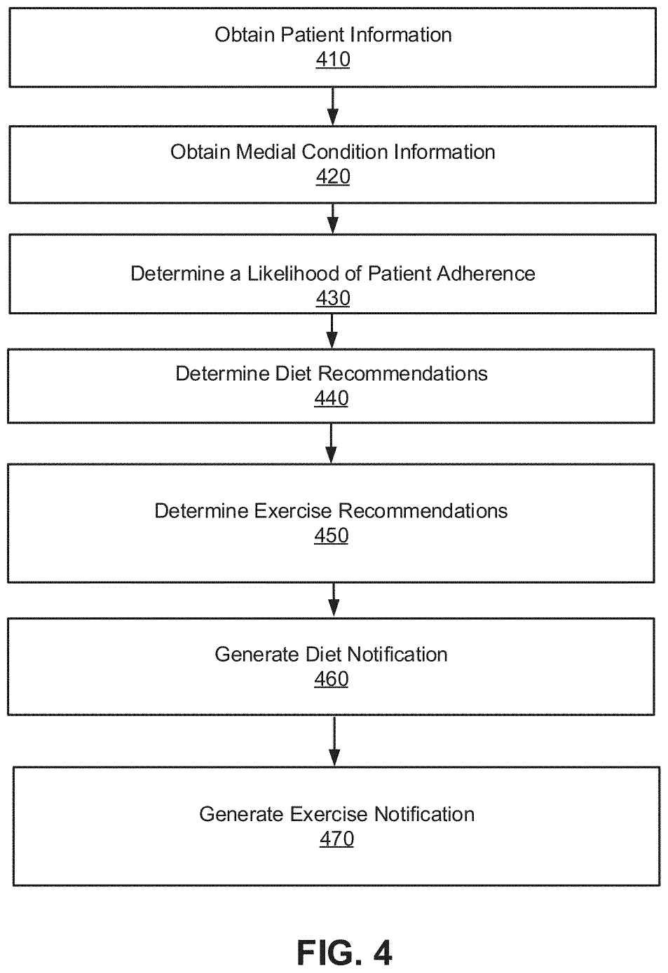

FIG. 4 illustrates a flow diagram describing a method for generating diet and exercise notifications based on the diet and exercise determinations made using the information provided by a user, in accordance with one embodiment. In some embodiments, method 400 can be implemented, for example, on a server system, e.g., treatment program server 120, as illustrated in FIGS. 1-2.

At operation 410, patient information component 106 of treatment program engine 126 obtains patient information. For example, patient information may include patient demographic information, patient preferences, and other relevant data.

At operation 420, medical information component 108 of treatment program engine 126 obtains patients' medical condition information. For example, medical condition information may include a type II diabetes mellitus diagnosis, blood glucose level, and other information related to one or more medical conditions.

At operation 430, patient adherence component 110 of treatment program engine 126 determines how likely a patient will follow a diet and exercise plan based on the patient information in step 410 and medical condition information in step 420, respectively.

At operation 440, diet determination component 112 determines a diet plan comprising one or more diet recommendations based on patient information in step 410, medical condition information in step 420, and a likelihood a patient will adhere to a diet in step 430. For example, the one or more diet recommendations may include daily meals comprising protein, fat, and carbohydrates amounts used for treating type II diabetes.

At operation 450, exercise determination component 114 determines an exercise plan comprising one or more exercise recommendations within a particular time period based on patient information in step 410, medical condition information in step 420, and a likelihood a patient will adhere to a diet in step 430. For example, the one or more exercise recommendations may include weekly aerobic and anaerobic exercises intended to induce weight loss in a patient.

At operation 460, notification component 116 generates one or more diet notifications based on one or more diet recommendations determined in step 440. At operation 470, notification component 116 generates one or more exercise notifications based on one or more exercise recommendations determined in step 450.

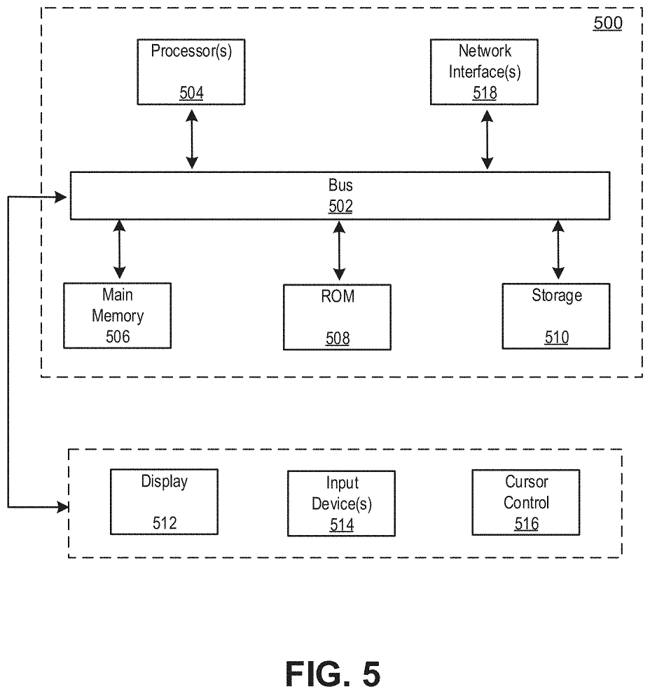

FIG. 5 depicts a block diagram of an example computer system 500 in which various embodiments described herein may be implemented. The computer system 500 includes a bus 502 or other communication mechanism for communicating information, one or more hardware processors 504 coupled with bus 502 for processing information. Hardware processor(s) 504 may be, for example, one or more general purpose microprocessors.

The computer system 500 also includes a main memory 505, such as a random access memory (RAM), cache and/or other dynamic storage devices, coupled to bus 502 for storing information and instructions to be executed by processor 504. Main memory 505 also may also be used for storing temporary variables or other intermediate information during execution of instructions to be executed by processor 504. Such instructions, when stored in storage media accessible to processor 504, render computer system 500 into a special-purpose machine that is customized to perform the operations specified in the instructions.

The computer system 500 further includes a read only memory (ROM) 508 or other static storage device coupled to bus 502 for storing static information and instructions for processor 504. A storage device 510, such as a magnetic disk, optical disk, or USB thumb drive (Flash drive), etc., is provided and coupled to bus 502 for storing information and instructions.

In general, the word “component,” “system,” “database,” and the like, as used herein, can refer to logic embodied in hardware or firmware, or to a collection of software instructions, possibly having entry and exit points, written in a programming language, such as, for example, Java, C or C++. A software component may be compiled and linked into an executable program, installed in a dynamic link library, or may be written in an interpreted programming language such as, for example, BASIC, Perl, or Python. It will be appreciated that software components may be callable from other components or from themselves, and/or may be invoked in response to detected events or interrupts. Software components configured for execution on computing devices may be provided on a computer readable medium, such as a compact disc, digital video disc, flash drive, magnetic disc, or any other tangible medium, or as a digital download (and may be originally stored in a compressed or installable format that requires installation, decompression or decryption prior to execution). Such software code may be stored, partially or fully, on a memory device of the executing computing device, for execution by the computing device. Software instructions may be embedded in firmware, such as an EPROM. It will be further appreciated that hardware components may be comprised of connected logic units, such as gates and flip-flops, and/or may be comprised of programmable units, such as programmable gate arrays or processors.

The computer system 500 may implement the techniques described herein using customized hard-wired logic, one or more ASICs or FPGAs, firmware and/or program logic which in combination with the computer system causes or programs computer system 500 to be a special-purpose machine. According to one embodiment, the techniques herein are performed by computer system 500 in response to processor(s) 504 executing one or more sequences of one or more instructions contained in main memory 505. Such instructions may be read into main memory 505 from another storage medium, such as storage device 510. Execution of the sequences of instructions contained in main memory 505 causes processor(s) 504 to perform the process steps described herein. In alternative embodiments, hard-wired circuitry may be used in place of or in combination with software instructions.

The term “non-transitory media,” and similar terms, as used herein refers to any media that store data and/or instructions that cause a machine to operate in a specific fashion. Such non-transitory media may comprise non-volatile media and/or volatile media. Non-volatile media includes, for example, optical or magnetic disks, such as storage device 510. Volatile media includes dynamic memory, such as main memory 505. Common forms of non-transitory media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, and networked versions of the same.

Non-transitory media is distinct from but may be used in conjunction with transmission media. Transmission media participates in transferring information between non-transitory media. For example, transmission media includes coaxial cables, copper wire and fiber optics, including the wires that comprise bus 502. Transmission media can also take the form of acoustic or light waves, such as those generated during radio-wave and infra-red data communications.

As used herein, the term “or” may be construed in either an inclusive or exclusive sense. Moreover, the description of resources, operations, or structures in the singular shall not be read to exclude the plural. Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps.

Terms and phrases used in this document, and variations thereof, unless otherwise expressly stated, should be construed as open ended as opposed to limiting. As examples of the foregoing, the term “including” should be read as meaning “including, without limitation” or the like. The term “example” is used to provide exemplary instances of the item in discussion, not an exhaustive or limiting list thereof. The terms “a” or “an” should be read as meaning “at least one,” “one or more” or the like. The presence of broadening words and phrases such as “one or more,” “at least,” “but not limited to” or other like phrases in some instances shall not be read to mean that the narrower case is intended or required in instances where such broadening phrases may be absent.

What is claimed is:

1. A method for treating a disorder associated with a disorder, comprising:
receiving patient demographic information associated with a patient;
receiving patient medical information associated with the patient;
determining a likelihood of patient adherence for the patient based upon the patient demographic information and the patient medical information;
generating a dietary regimen comprising one or more meal recommendations consumed by the patient during a time period and an exercise regimen comprising one or more exercise recommendations performed by the patient during the time period based on the likelihood of patient adherence.

2. The method of claim 1, wherein the patient demographic information includes age, gender, and marital status.

3. The method of claim 1, wherein the patient medical information includes at least one medical condition associated with the patient.

4. The method of claim 1, further comprising obtaining information related to other patients based on the patient demographic information and the patient medical information.

5. The method of claim 4, wherein the determination of likelihood of patient adherence for the patient is based upon the information related to other patients.

6. The method of claim 4, wherein the determination of the likelihood of patient adherence of the user is calculated using Bayesian statistics.

7. The method of claim 1, wherein the meal recommendations comprise at least one meal from a restaurant.

8. The method of claim 1, wherein a total daily caloric intake of the one or more meal recommendations is less than a baseline caloric intake for the patient.

9. The method of claim 1, wherein a daily carbohydrate amount of the one or more meal recommendations is approximately less than 50 grams.

10. The method of claim 1, wherein the disorder includes at least a metabolic syndrome, insulin resistance, and diabetes.
